(19) United States
(12) Patent Application Publication
(10) Pub. No.: US 2022/0044106 A1
(43) Pub. Date: Feb. 10, 2022

(54) ATTENTION ENCODING STACK IN AGGREGATION OF DATA

(71) Applicant: X Development LLC, Mountain View, CA (US)

(72) Inventors: Mustafa Ispir, Mountain View, CA (US); Edward Michel F De Brouwer, San Francisco, CA (US); Pramod Gupta, Mountain View, CA (US); Garrett Raymond Honke, Mountain View, CA (US); Vladimir Miskovic, Binghamton, NY (US)

(21) Appl. No.: 16/986,714

(22) Filed: Aug. 6, 2020

(51) Int. Cl. G06N 3/08; G16H 70/60; G16H 50/20

(52) U.S. Cl. CPC G06N 3/08; G16H 50/20; G16H 70/60

(57) ABSTRACT

A machine learning system for aggregating electroencephalographic (EEG) data, as well as external data, in preparation for downstream analysis via further machine learning models. Machine learning models can be used to assist in diagnosis of various mental health conditions, brain-computer interface, mood detection systems, or other biometric functions. Implementations of the present disclosure, employ a portion of the transformer network (the attention encoder stack) to aggregate EEG trials or EEG data segments, in a data-driven way, by ensuring the important content of each trial is not lost. Each EEG trial to be aggregated is converted into an input embedding, or a vector which numerically represents the data in the trial.

### TECHNICAL FIELD

This disclosure generally relates to using machine learning methods to aggregate EEG trial embeddings as well as data from other sources into an aggregate embedding.

### BACKGROUND

In some machine learning processes, a large number of EEG trials are recorded for a single individual, and then aggregated using an averaging method into a single representative EEG to be used as an input to a machine learning model. In these implementations, it is possible that important data useful for diagnosing an individual's mental health is removed during the averaging. Therefore, an improved method for aggregating multiple EEG trials is desired.

### SUMMARY

In general, the disclosure relates to a machine learning system for aggregating electroencephalogram (EEG) trials in preparation for downstream analysis via further machine learning models. A machine learning model can be used to assist in diagnosis of various mental health conditions, however an input to this diagnosis model must be succinct enough to be computationally feasible, yet still contain all necessary relevant information. Additional information from other sensors can also be incorporated into the machine learning algorithm and can provide further contextual information. For example, an individual can wear a heart rate monitor before or during the EEG trial(s), as well as fill out a questionnaire before each EEG trial. It is difficult for computing systems to process these types of additional information (e.g., heart rate data and questionnaire responses) in conjunction with EEG signals. For instance it can be difficult for a computing system to extract relevant aspects of such additional information when compared to the EEG data. Therefore, the present disclosure provides a solution for combining this additional data with the EEG data, in a manner which reduces irrelevant aspects of data while retaining relevant data.

An attention encoder stack (AES) network can be used to aggregate EEG trials and additional external data in a data-driven way, by ensuring the important content of each trial or data set is not lost. Each EEG trial to be aggregated is converted into a trial embedding, or a vector which numerically represents the data in the trial. In some implementations, the embeddings for all EEG trials are the same length (e.g., 512, 1024, etc.). Further, each set of additional data from an additional sensor (e.g., heart rate monitor, questionnaire, sleep monitor, activity sensor, pedometer, ect.) can be converted into an external embedding. The trial embeddings and the external embeddings together for a set of input embeddings, which can then be used as input to an attention encoder stack network, which uses a self-attention model to determine an output embedding that accurately represents an aggregation of the inputs, retaining important data and filtering noise. The attention function can either be a scaled dot product attention function, or a multi-head attention function.

In general, innovative aspects of the subject matter described in this specification can be embodied in a system that conducts the actions including: identifying two or more input embeddings that are a vector of length n and represent an EEG trail of an individual. Identifying at least one external embedding which is a vector of length n representing data associated with the individual that is not from an EEG trial. The two or more input embeddings and the at one or more external embeddings are encoded using an attention encoder stack network to generate an output embedding that represents an aggregation of the two or more input embeddings. The output embedding is a vector of fixed length k. The output embedding is provided as input to a neural network to determine a mental health status of the individual. These and other implementations can each optionally include one or more of the following features.

In some implementations, the attention encoder stack includes a plurality of encoder layers in a series, the first encoder layer receiving the input embedding and sending its output to the next encoder in the series, and the final encoder in the series outputting the output embedding. Each encoder layer in the series includes (1) a first sublayer including a multi-head attention network, (2) a second sublayer including a feed forward network, and (3) residual connection which receive an input vector for each sublayer and add it to an output vector of each sublayer, then normalize the resulting vector.

In some implementations, the multi-head attention network comprises a plurality of scaled dot-product attention networks, each scaled dot-product attention network using a unique parameter matrix.

In some implementations, the attention encoder stack includes six encoder layers in series.

In some implementations, each input embedding is a vector of unit values from the penultimate layer of a convolutional neural network processing EEG data.

In some implementations, the fixed vector of length k has a length of 512.

In some implementations, determining a mental health status of the individual includes diagnosing a mental health disorder.

In some implementations, the EEG trial of the individual was recorded while the individual was presented with stimuli.

In some implementations, the external embeddings include data from at least one of a heart rate monitor, a sleep monitor, a questionnaire, an activity sensor, a pedometer, or any suitable combination thereof.

Particular implementations of the subject matter described in this specification can be implemented so as to realize one or more of the following advantages. For example, one advantage of using a transformer network to aggregate EEG trials is that each input embedding can be the same length regardless of the length of the trial. Therefore the transformer network can readily aggregate multiple trials of different lengths. Another advantage is the transformer can readily accept embeddings that are not from EEG trials. Therefore embeddings from additional sensors or external data (e.g., questionnaires, wearables, or other information) can be readily incorporated and impact the aggregate output.

The details of one or more implementations of the subject matter described in this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts an example of a system architecture for EEG analysis.

FIG. 2 depicts an example of an Attention Encoder Stack (AES) network for use in aggregating EEG trials according to implementations of the present disclosure.

FIG. 3 is a flow diagram depicting an example method for aggregating EEG trials using an Attention Encoder Stack (AES).

FIG. 4 is a schematic diagram of a computer system.

### DETAILED DESCRIPTION

The disclosure generally relates to a machine learning system for aggregating electroencephalographic (EEG) data and external data in preparation for downstream analysis via further machine learning models. Machine learning models can be used to assist in diagnosis of various mental health conditions, brain-computer interface, mood detection systems, or other biometric functions. However inputs to such a diagnosis model must be succinct enough to be computationally feasible, yet still contain all necessary relevant information. Implementations of the present disclosure, employ a portion of the transformer network (the attention encoder stack) to aggregate EEG trials or EEG data segments, and data from external sources in a data-driven way, by ensuring the important content of each trial or dataset is not lost. Each EEG trial to be aggregated is converted into a trial embedding, or a vector which numerically represents the data in the trial. In some implementations, the embeddings for all EEG trials are the same length (e.g., 512, 1024, etc.). External datasets can also be converted to embeddings. External datasets can include, for example, data from a sleep monitor, or a heart rate sensor, a pedometer, or other activity sensor. Additionally external dataset can include results or data from a survey or questionnaire provided for an individual to complete. Both the EEG trial embeddings and the external embeddings combined represent a set of input embeddings for an aggregation process. For example, the input embeddings can be used as input to the transformer network, which uses a self-attention model to determine an output embedding that accurately represents an aggregation of the input trials, retaining important data and filtering noise. The attention function can either be a scaled dot product attention function, or a multi-head attention function.

In some implementations, a series of self-attention point-wise encoders, the Attention Encoder Stack (AES) can be used to aggregate input embeddings in an intelligent way by ensuring the important content of each trial or dataset is not lost. Each dataset to be aggregated is converted into an input embedding (either a trial embedding if the dataset is an EEG trial, or an external embedding if the dataset is from an external source), or a vector which numerically represents the data in the trial. In some implementations, all input embeddings are the same length (e.g., 512, 1024, etc.). The input embeddings can then be used as input to the AES. The AES uses a self-attention model to determine an output embedding that accurately represents an aggregation of the input trials, retaining data associated with brain activity and filtering noise. The attention function can either be a scaled dot product attention function, or a multi-head attention function.

EEG's trials can have a large amount of noise, making extracting the useful data difficult for a machine learning process. Additionally, different EEG trials may not have a consistent signal to noise ratio (e.g., one trial may have significantly more useful information while another trial may have significantly more noise when compared to the average). Therefore, it is desirable to aggregate multiple EEG trials in a way which preserves information from trials which contain more information associated with brain activity while filtering or reducing noise from trials which have a lower signal to noise ratio. Additionally different external sensors and datasets can have varying levels of relevance to an individual's mental health. For example, some heartrate datasets can correspond strongly with an individual's physical exertion and mental state, but other heartrate datasets may be noisy and have little correspondence (e.g., the sensor was worn improperly). Advantageously, the AES allows the generation of an aggregate dataset which includes information determined to be more important for diagnosis by the self-attention model, instead of putting an equal weight on each trial or dataset as is done when averaging.

Since the AES is that it can readily accept embeddings that are not from EEG trials. Therefore embeddings from additional sensors or external data (e.g., questionnaires, wearables, or other information) can be readily incorporated and impact the aggregate output. The AES can learn which information should be weighted more heavily, and is more relevant to a mental health diagnosis than other, less relevant information. Further, each individual may have a differing number of available EEG trials or other datasets associated with them. By aggregating all of a particular individual's data into a single representative dataset, it is possible to perform machine learning on a multitude of individuals, without underrepresenting individuals who have less data available, as each individual contributes a single (or multiple, set number of) aggregate datasets to the corpus of data to be used in training the machine learning model.

An EEG trial can include providing diagnostic content for presentation to the individual. During the presentation of the diagnostic content to the individual, the EEG signals representing the individual's neuro-electrical activity from an EEG sensor system are recorded. In general, any sensors capable of detecting neuro-electrical activity may be used. For example, the neuro-electrical activity sensors can be one or more individual electrodes (e.g., multiple EEG electrodes) that are connected by wired connection. Example neuro-electrical activity sensor systems can include, but are not limited to, EEG systems, a wearable neuro-electrical activity detection device, a magnetoencephalography (MEG) system, and an Event-Related Optical Signal (EROS) system, sometimes also referred to as “Fast NIRS” (Near Infrared spectroscopy). A neuro-electrical activity sensor system can transmit neuro-electrical activity data to form an EEG trial.

A content presentation system is configured to present content to the individual for each diagnostic trial while the individual's neuro-electrical activity is measured during the diagnostic testing. For example, the content presentation system can be a multimedia device, such as a desktop computer, a laptop computer, a tablet computer, or another multimedia device. Further, the content presentation system can receive input from the individual and apply the input to the EEG trial.

The EEG trial data represents EEG data of an individual's neuro-electrical activity while the individual is presented with diagnostic content that is designed to trigger responses in particular brain systems, e.g., a brain system related to depression. During a diagnostic test, for example, an individual may be presented with diagnostic content during several trials. Each trial can include diagnostic content with stimuli designed to trigger responses in one particular brain system or multiple different brain systems. As one example a trial could include diagnostic content with physically active tasks for an individual to perform in order to achieve a reward so as to stimulate the dopaminergic reward system in the brain.

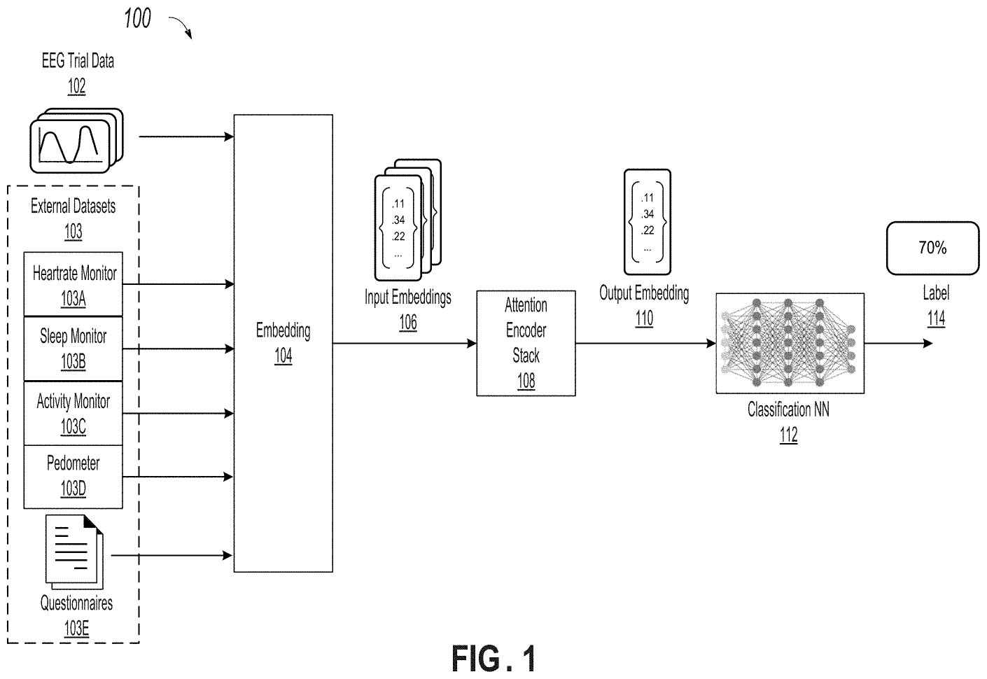

FIG. 1 depicts an overall system architecture for EEG analysis. The system 100 receives EEG trial data 102 and external datasets 103. The EEG trial data 102 can be digital representations of analog measurements taken during an EEG trial during which an individual is presented with various stimuli. For example, stimulus intended to trigger particular responses in portions of the brain, such as the visual cortical system, or the anterior cingulate cortex, can be presented to an individual and the corresponding neuro-electrical activity response recorded in the EEG trial can be marked or labeled, and associated with a timestamp of when the stimulus was presented. Each set of EEG trial data 102 can be provided as input to an embedding process 104. The stimulus can include, but is not limited to, visual content such as images or video, audio content, interactive content such as a game, or a combination thereof. For example, emotional content (e.g., a crying baby; a happy family) can be configured to probe the brain's response to emotional images. As another example, visual attentive content can be configured to measure the brain's response to the presentation of visual stimuli. Visual attentive content can include, e.g., the presentation of a series of images that change between generally positive or neutral images and negative or alarming images. For example, a set of positive/neutral images (e.g., images of a stapler, glass, paper, pen, glasses, etc.) can be presented with negative/alarming images (e.g., a frightening image) interspersed there between. The images can be presented randomly or in a pre-selected sequence. Moreover, the images can alternate or “flicker” at a predefined rate. As another example, error monitoring content can be used to measure the brain's response to making mistakes. Error monitoring content can include, but is not limited to, interactive content designed to elicit decisions from an individual in a manner that is likely to result in erroneous decisions. For example, the interactive content can include a test using images of arrows and require the individual to select which direction the arrow(s) is/are pointing, but may require the decisions to be made quickly so that the user will make errors. In some implementations, no content is presented, e.g., in order to measure the brain's resting state and obtain resting state neuro-electrical activity.

External datasets 103 are datasets that are separate from the EEG trial data. They can include, but are not limited to, data recorded from a heart rate monitor 103A, a sleep monitor 103B, an activity monitor 103C, a pedometer 103D, and one or more questionnaires 103E. The external datasets can, in some cases, be collected in between EEG trials where EEG trial data 102 is collected. For example, if an individual receives an EEG trial once per week, they can additionally be asked to wear a sleep monitor every night in between trials. In some implementations external datasets 103 can be collected simultaneously with EEG trial data 102. For example, when an EEG trial is occurring, the individual can wear a heart rate monitor providing heart rate data 103A during the trial. This heart rate data 103A can largely correlate to a particular set of EEG trial data 102, and can show information corresponding to certain stimulus provided to the individual during an EEG trial. In some implementations, individuals can use a wearable data collection device (e.g., a smart watch, or other necklace or other wearable device) which collects data (e.g., sleep, activity, heart rate etc.) continuously for a period of time (e.g., days, weeks, etc.). Additionally individuals can complete surveys or questionnaires 103E periodically (e.g., daily) designed to determine information about their current mental state.

The embedding process 104 converts the raw EEG trial data 102 and the external datasets 103 into vectors of fixed length. Resulting in an input embedding 106 for each set of EEG trial data 102 and each external dataset 103. In some implementations, the embedding process 104 is a convolutional neural network (CNN) that is trained simultaneously with the rest of the neural networks in system 100. The embedding process 104 can accept analog or digital data from each set of EEG trial data 102, as well as additional data such as metadata (e.g., timestamps, manual data tagging, etc.). In some implementations, the embedding process 104 is a part of an upstream CNN performing additional or external analysis on the EEG trial data 102 and external datasets 103. In these implementations, while the final or output layer of the upstream CNN can be used for separate analysis, each unit in the penultimate layer of the CNN is used in the embedding process 104. These units each have a value which can be mapped to a vector representing the input embedding 106 which is to be the output of the embedding process 104. In some implementations the embedding process is a principle component analysis (PCA) or matrix factorization technique. In some implementations the embedding process is a separate neural network, such as a variational autoencoder. In some implementations, data from each source (e.g., EEG trial data 102, and external datasets 103) are embedded separately, using unique processes. For example, heart rate data can be embedded using a time sampled version of the analog heart rate data.

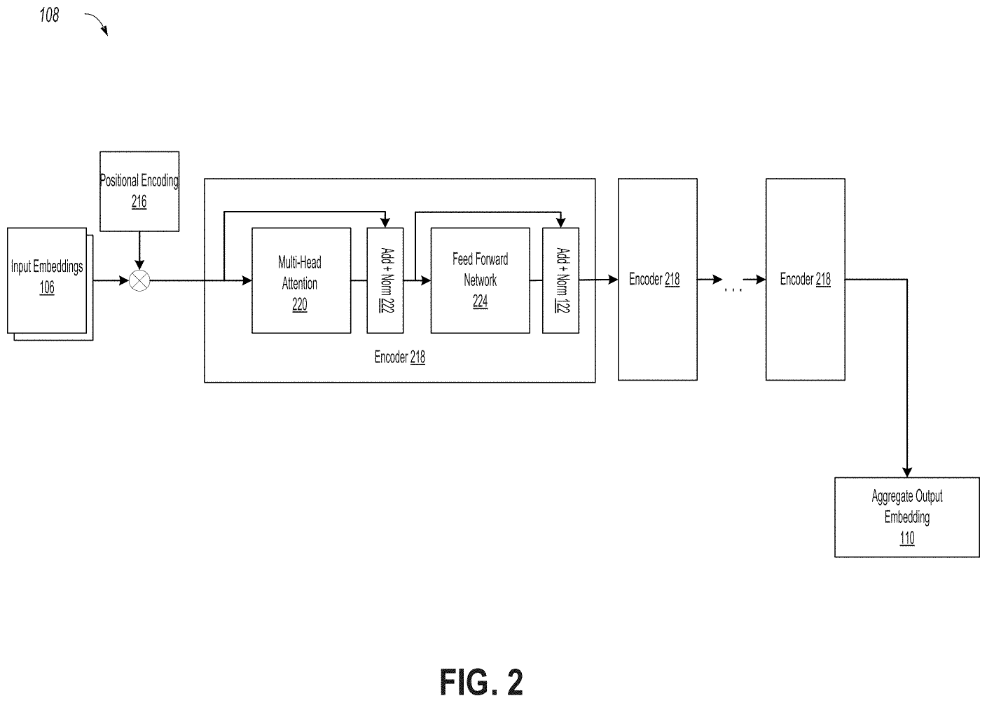

Multiple input embeddings 106, each associated with a particular individual, are then accepted by the Attention Encoder Stack (AES) 108. The AES can be similar to the encoder portion of a transformer network. The AES 108, which is described in further detail below with reference to FIG. 2, aggregates the multiple input embeddings 106 to form a single output embedding 110. The output embedding 110 is more than merely an averaging of the input embeddings 106, but is a representative embedding which retains relevant information from the input embeddings 106 while filtering noise or irrelevant information.

The output embedding 110 can then be used for further analysis/classification, e.g., using a classification neural network 112. Each output embedding 110 can be an aggregate that is representative of the mental state of a particular individual over a number of trials (e.g., 60 or 100, etc.) and external datasets. The output embedding 110 can be analyzed by a classification neural network 112 which can label, or otherwise provide a diagnosis based on the output embedding 110. In some implementations, the classification neural network 112 can be a feedforward autoencoder neural network. For example, the classification neural network 112 can be a three-layer autoencoder neural network. The classification neural network 112 may include an input layer, a hidden layer, and an output layer. In some implementations, the neural network has no recurrent connections between layers. Each layer of the neural network may be fully connected to the next, e.g., there may be no pruning between the layers. The classification neural network 112 can include an optimizer for training the network and computing updated layer weights, such as, but not limited to, ADAM, Adagrad, Adadelta, RMSprop, Stochastic Gradient Descent (SGD), or SGD with momentum. In some implementations, the classification neural network 112 may apply a mathematical transformation, e.g., a convolutional transformation or factor analysis to input data prior to feeding the input data to the network.

In some implementations, the classification neural network 112 can be a supervised model. For example, for each input provided to the model during training, the classification neural network 112 can be instructed as to what the correct output should be. The classification neural network 112 can use batch training, e.g., training on a subset of examples before each adjustment, instead of the entire available set of examples. This may improve the efficiency of training the model and may improve the generalizability of the model. The classification neural network 112 may use folded cross-validation. For example, some fraction (the “fold”) of the data available for training can be left out of training and used in a later testing phase to confirm how well the model generalizes. In some implementations, the classification neural network 112 may be an unsupervised model. For example, the model may adjust itself based on mathematical distances between examples rather than based on feedback on its performance.

In some examples, the classification neural network 112 can provide a binary output label 114, e.g., a yes or no indication of whether the individual is likely to have a particular mental disorder. In some examples, the classification neural network 112 provides a score label 114 indicating a likelihood that the individual has one or more particular mental conditions. In some examples, the classification neural network 112 can provide a severity score indicating how severe the predicted mental condition is likely to be, for example, with respect to the individual's overall quality of life. In some implementations, the classification neural network 112 sends output data indicating the individual's likelihood of experiencing a particular mental condition to a user computing device. For example, the classification neural network 112 can send its output to a user computing device associated with the individual's doctor, nurse, or other case worker.

FIG. 2 is a detailed diagram describing the AES network 108. The AES accepts multiple input embeddings 106 and provides a single output embedding 110, which is an aggregation of the input embeddings 106. The AES includes two or more encoders 218. In some implementations, six encoders are used. Each encoder receives the output from the previous encoders, and provides it output to the next encoder in the stack.

The encoders 218, at a high level, receive a number of embeddings, and convert each embedding into a query vector, a key vector, and a value vector, by multiplying each embedding received with a weight matrix that is set during model training. Each weight matrix can be unique, resulting in unique query, key, and value vectors. Each of the key, query, and value vectors can be used in a scaled dot-product attention algorithm which results in an output attention vector for each embedding. The attention vector can be calculated as

$$Attention ( Q , W , V ) = softmax ( \frac{{QK}^{T}}{\sqrt{{d}_{k}}} ) V ,$$

where Q, K, and V are the query, key, and value vectors and dₖ is the dimension of the key vector. The softmax( ) function is a normalized exponential function. This can be done multiple times in parallel for each input embedding, and is done by the multi-head attention network 220. The multi-head attention network 220 outputs an attention vector for each head. These attention vectors can then be concatenated and multiplied by an additional weight matrix to yield a single attention vector for each received embedding which includes information from each head of the multi-head attention network 220. This single attention vector can be combined and normalized with a residual connection. For example, the received embeddings can be added to their associated attention vectors and normalized to improve network stability. These residual connections are shown in FIG. 2 as add+norm blocks 222. The attention vectors with their residual connections included can then be used as input to a feed forward network 224. The feed forward network 224 can be, for example, a three layer neural network that outputs a vector in a format suitable to be ingested by the next encoder 218 in the stack.

In some implementations, the input embeddings can be multiplied by a positional encoding function 216. This can be a sinusoid, or other function (e.g., exponentially decaying sinusoidal function) which imparts a value associated with the relative position of each embedding in the sequence of embeddings. Each encoder 218 receives the entire set of embeddings simultaneously. This is advantageous as it allows for parallelization in the computational process, however if the sequence of embeddings, or the position of each embedding in the set of embeddings has value, it can be accounted for using positional encoding. In some implementations, the positional encoding function 216, which is used to define the value for which each embedding is to be multiplied is similar to the following equations:

$${PE}_{( pos , 2 i )} = sin ( \frac{pos}{{10000}^{\frac{2 i}{{d}_{model}}}} ) and {PE}_{( pos , 2 i + 1 )} = cos ( \frac{pos}{{10000}^{\frac{2 i}{{d}_{model}}}} ) ,$$

where pos represents the position and i is the dimension of the embeddings, while d_{model} is the dimension of the AES 108. This multiplication embeds positional information in the input embeddings 106 prior to them being processed by the first encoder 218. This can allow the AES 108 to distinguish between input embeddings 106 from EEG trial data 102, and input embeddings 106 from external datasets 103. By providing all of the data simultaneously, with positional encoding to the AES 108, the AES 108 can learn via self-attention which data is most relevant to the task (e.g., diagnosis), it has been trained to perform.

The final encoder 218 in the encoder stack can output a single vector which represents an aggregate output embedding 110. In some implementations the aggregate output embedding 110 is the final vector produced by the final encoder 218. In some implementations, the aggregate output embedding 110 is a combination of output vectors produced by the final encoder 218. The output embedding 110 is a combination of all the input embeddings 106, and is weighted based on the attention layers in each encoder 218 such that it includes useful information while excluding noise or bad information.

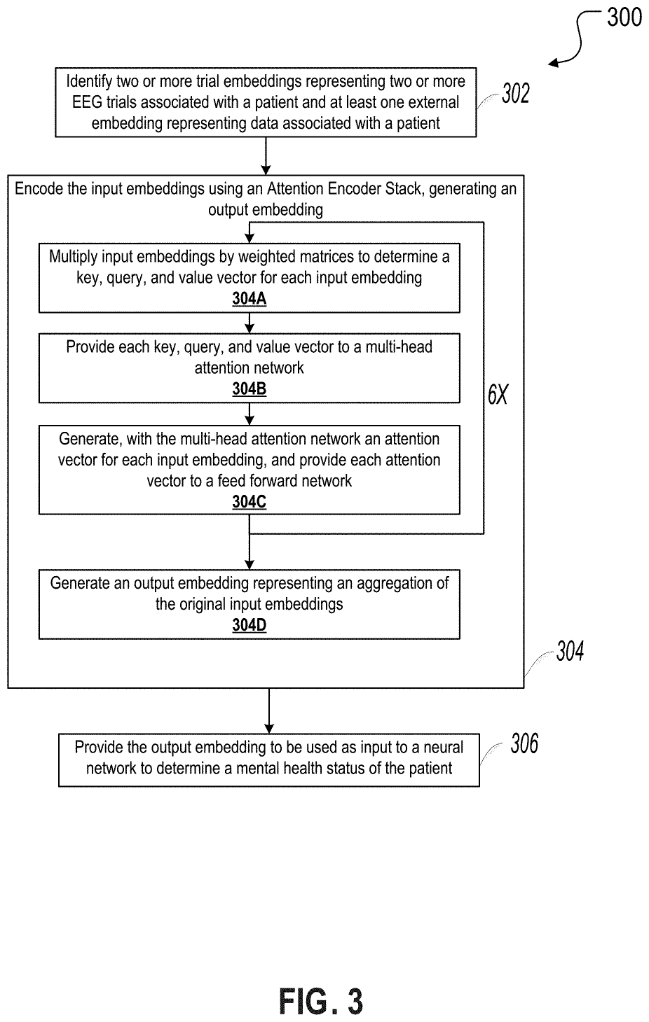

FIG. 3 is a flow diagram of an example process 300 for aggregating EEG trials using an AES. However, it will be understood that process 300 may be performed, for example, by any suitable system, environment, software, and hardware, or a combination of systems, environments, software, and hardware as appropriate. In some instances, process 300 can be performed by the system for aggregating EEG trials as described in FIG. 1, or portions thereof, and further described in FIG. 2, as well as other components or functionality described in other portions of this description. In other instances, process 300 may be performed by a plurality of connected components or systems. Any suitable system(s), architecture(s), or application(s) can be used to perform the illustrated operations.

At 302, two or more input embeddings are identified to be aggregated. An input embedding can be trial embedding, or an external embedding, both of which are a vector representation of an EEG trial or other data associated with an individual. A single individual may have multiple trials (e.g., 50 or 100, etc.) each trial having a varying amount of noise and information and a varying quality. Additionally a single individual may have multiple external datasets, which can be related to the trials, or independent of the trials. The input embeddings need to be aggregated in a way that preserves the useful information and presents a high quality aggregate embedding that is representative of the individuals mental health, such that a downstream machine learning algorithm can provide useful information about a mental health status of the individual (e.g., a diagnosis, probability of a mental disorder, or probability of the individual experiencing future disorders).

At 304, the input embeddings are encoded using an AES to generate an output embedding which is aggregate of the input embeddings. At 304A each input embedding is multiplied three separate weight matrices, each multiplication resulting in a key vector, query vector, and a value vector. The key, query, and value vectors for each embedding are then provided to a multi-head attention network at 304B.

The multi-head attention network can, for each set of key, query, and value vectors, generate, for each head, an attention vector that indicates portions of the input embedding which are more important than other portions. Because each head of the multi-head attention network generates a separate attention vector for each input embedding, and only a single attention vector is expected in the following processes, the attention vectors can be concatenated, then multiplied by an additional weight matrix to yield a single attention vector, (with attention information from each head of the multi-head attention network), for each input embedding. At 304C, these combined attention vectors are then provided to a feed forward network which, using the attention vectors, can generate a set of input embeddings to be consumed by the following attention encoder in the AES. In some implementations, 304A through 304C is repeated. For example, 304A through 304C can be repeated six times, or more. In some implementations 304A through 304C are not repeated, and process 300 proceeds directly to 304D.

At 304D the feed forward network of the final encoder in the AES generates a single output embedding, which is an aggregation of the input embeddings. In some implementations the output embedding is the final vector produced by the final encoder. In some implementations, the output embedding is a combination of output vectors produced by the final encoder. The output embedding is a combination of all the input embeddings, and is weighted based on the attention vectors in each encoder such that it includes useful information while excluding noise or bad information.

At 306, the output embedding can be provided to a machine learning algorithm (e.g., neural network) to determine a mental health status of the individual. This can be a classification neural network similar to classification neural network 112 as discussed with reference to FIG. 1. The machine learning algorithm can label, or otherwise provide a diagnosis based on the output embedding.

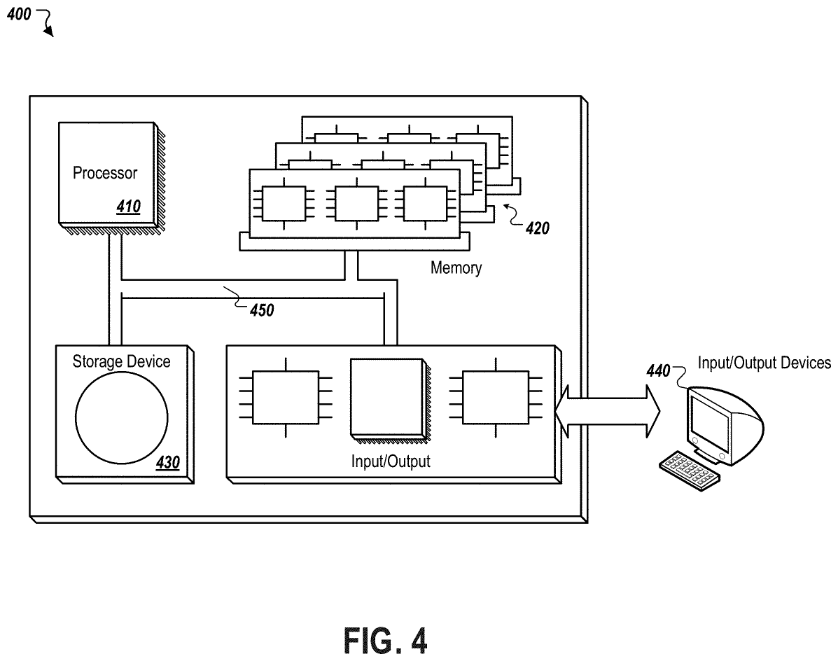

FIG. 4 is a schematic diagram of a computer system 400. The system 400 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 400) and their structural equivalents, or in combinations of one or more of them. The system 400 is intended to include various forms of digital computers, such as laptops, desktops, workstations, personal digital assistants, servers, blade servers, mainframes, and other appropriate computers, including vehicles installed on base units or pod units of modular vehicles. The system 400 can also include mobile devices, such as personal digital assistants, cellular telephones, smartphones, and similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Further to the descriptions above, a user may be provided with controls allowing the user to make an election as to both if and when systems, programs, or features described herein may enable collection of user information. In addition, certain data may be treated in one or more ways before it is stored or used, so that personally identifiable information is removed. For example, a user's identity may be treated so that no personally identifiable information can be determined for the user, or a user's skin data and/or diagnosis cannot be identified as being associated with the user. Thus, the user may have control over what information is collected about the user and how that information is used

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A computer-implemented method executed by one or more processors and comprising:
identifying a plurality of trial embeddings, wherein a trial embedding is a vector of length n representing an electroencephalogram (EEG) trial of an individual;
identifying at least one external embedding, wherein an external embedding is a vector of length n representing data associated with the individual that is not from an EEG trial, and wherein the at least one external embeddings and the plurality of trial embeddings form a plurality of input embeddings;
encoding the plurality of input embeddings using an attention encoder stack network to generate an output embedding that represents an aggregation of the plurality of input embeddings, wherein the output embedding is a vector of fixed length k; and
providing the output embedding to be used as input in a neural network to determine a mental health status of the individual.

2. The method of claim 1, wherein the attention encoder stack comprises:
a plurality of encoder layers in a series, with a first encoder layer receiving each input embedding and sending its output to the next encoder in the series with a final encoder in the series outputting the output embedding, wherein each encoder layer comprises:
a first sublayer comprising a multi-head attention network;
a second sublayer comprising a feed forward network; and
residual connections which take an input vector of each sublayer and add it to an output vector of each sublayer, then normalize a resulting vector.

3. The method of claim 2, wherein the multi-head attention network comprises a plurality of scaled dot-product attention networks, each scaled dot-product attention network using a unique parameter matrix.

4. The method of claim 2, wherein the plurality of encoder layers comprise six encoder layers.

5. The method of claim 1, wherein each trial embedding is a vector of unit values of a penultimate layer of a convolutional neural network processing EEG data.

6. The method of claim 1, wherein the fixed vector of length k has a length of 512.

7. The method of claim 1, wherein the external embeddings comprise data from at least one of:
a heart rate monitor;
a sleep monitor;
a questionnaire;
an activity sensor; or
a pedometer.

8. The method of claim 1, wherein determining a mental health status of the individual includes diagnosing a mental health disorder.

9. A system for aggregating data, comprising:
one or more processors;
one or more tangible, non-transitory media operably connectable to the one or more processors and storing instructions that, when executed, cause the one or more processors to perform operations comprising:
identifying a plurality of trial embeddings, wherein a trial embedding is a vector of length n representing an electroencephalogram (EEG) trial of an individual;
identifying at least one external embedding, wherein an external embedding is a vector of length n representing data associated with the individual that is not from an EEG trial, and wherein the at least one external embeddings and the plurality of trial embeddings form a plurality of input embeddings;
encoding the plurality of input embeddings using an attention encoder stack network to generate an output embedding that represents an aggregation of the plurality of input embeddings, wherein the output embedding is a vector of fixed length k; and
providing the output embedding to be used as input in a neural network to determine a mental health status of the individual.

10. The system of claim 9, wherein the attention encoder stack comprises:
a plurality of encoder layers in a series, with a first encoder layer receiving each input embedding and sending its output to the next encoder in the series with a final encoder in the series outputting the output embedding, wherein each encoder layer comprises:
a first sublayer comprising a multi-head attention network;
a second sublayer comprising a feed forward network; and
residual connections which take an input vector of each sublayer and add it to an output vector of each sublayer, then normalize a resulting vector.

11. The system of claim 10, wherein the multi-head attention network comprises a plurality of scaled dot-product attention networks, each scaled dot-product attention network using a unique parameter matrix.

12. The system of claim 10, wherein the plurality of encoder layers comprise six encoder layers.

13. The system of claim 9, wherein each trial embedding is a vector of unit values of a penultimate layer of a convolutional neural network processing EEG data.

14. The system of claim 9, wherein the fixed vector of length k has a length of 512.

15. The system of claim 9, wherein the external embeddings comprise data from at least one of:
a heart rate monitor;
a sleep monitor;
a questionnaire;
an activity sensor; or
a pedometer.

16. A non-transitory computer readable storage medium storing instructions that, when executed by at least one processor, cause the at least one processor to perform operations comprising:
identifying a plurality of trial embeddings, wherein a trial embedding is a vector of length n representing an electroencephalogram (EEG) trial of an individual;
identifying at least one external embedding, wherein an external embedding is a vector of length n representing data associated with the individual that is not from an EEG trial, and wherein the at least one external embeddings and the plurality of trial embeddings form a plurality of input embeddings;
encoding the plurality of input embeddings using an attention encoder stack network to generate an output embedding that represents an aggregation of the plurality of input embeddings, wherein the output embedding is a vector of fixed length k; and
providing the output embedding to be used as input in a neural network to determine a mental health status of the individual.

17. The medium of claim 16, wherein the attention encoder stack comprises:
a plurality of encoder layers in a series, with a first encoder layer receiving each input embedding and sending its output to the next encoder in the series with a final encoder in the series outputting the output embedding, wherein each encoder layer comprises:
a first sublayer comprising a multi-head attention network;
a second sublayer comprising a feed forward network; and
residual connections which take an input vector of each sublayer and add it to an output vector of each sublayer, then normalize a resulting vector.

18. The medium of claim 17, wherein the multi-head attention network comprises a plurality of scaled dot-product attention networks, each scaled dot-product attention network using a unique parameter matrix.

19. The medium of claim 16, wherein each trial embedding is a vector of unit values of a penultimate layer of a convolutional neural network processing EEG data.

20. The medium of claim 16, wherein the external embeddings comprise data from at least one of:
a heart rate monitor;
a sleep monitor;
a questionnaire;
an activity sensor; or
a pedometer.
